(19) United States
(12) Patent Application Publication
(10) Pub. No.: US 2025/0375321 A1
(43) Pub. Date: Dec. 11, 2025

(54) DIAPER PANTS HAVING COMPONENTS WITH SOLID COLORED NONWOVEN WEBS AND FILMS

(71) Applicant: The Procter & Gamble Company, Cincinnati, OH (US)

(72) Inventors: Christopher Scott CAMERON, Cincinnati, OH (US); Sarah Nicole WOLFE, Cincinnati, OH (US); Abhishek SURUSHE, Mason, OH (US); Koichi MORIMOTO, Cincinnati, OH (US); J Michael BILLS, Mason, OH (US); Jeromy Thomas RAYCHECK, South Lebanon, OH (US); Uwe SCHNEIDER, Cincinnati, OH (US); Michael Devin LONG, Hamilton Twp, OH (US)

(21) Appl. No.: 19/220,173

(22) Filed: May 28, 2025

Related U.S. Application Data
(60) Provisional application No. 63/656,764, filed on Jun. 6, 2024.
(60) Provisional application No. 63/683,308, filed on Aug. 15, 2024.

(51) Int. Cl. A61F 13/15; A61F 13/49

(52) U.S. Cl. CPC A61F 13/15203; A61F 13/49011; A61F 13/4902

(57) ABSTRACT

The present disclosure relates to absorbent articles including absorbent chassis, belts, and waistbands, and more particularly, to diaper pants comprising solid colored substrates, such as nonwoven webs and/or films, combined so as to provide relatively stark color contrasts between waistbands and belts and relatively subtle color contrasts between absorbent chassis and belts. The substrates of the present disclosure, such as nonwovens and films, may comprise a colorant used to create color in a substrate. Such substrates may be used to manufacture portions of an absorbent article, such as components comprising a laminate, that are consumer pleasing and premium looking.

### CROSS REFERENCE TO RELATED APPLICATION

This application claims the benefit of U.S. Provisional Application No. 63/683,308, filed Aug. 15, 2024 and U.S. Provisional Application No. 63/656,764, filed on Jun. 6, 2024, which are incorporated by reference herein.

### FIELD

The present disclosure relates to absorbent articles including absorbent chassis, belts, and waistbands, and more particularly, to diaper pants comprising solid colored nonwoven webs and/or films combined so as to provide relatively stark color contrasts between waistbands and belts and relatively subtle color contrasts between absorbent chassis and belts.

### BACKGROUND

Along an assembly line, diaper pants and other disposable absorbent articles may be assembled by adding components to and otherwise modifying advancing, continuous webs of material. Webs of material and component parts used to manufacture diapers may include: backsheets, topsheets, absorbent cores, and various types of elastic webs and components such as belts and waistbands.

Some consumers may prefer purchasing absorbent articles, such as diaper pants, having various types of different designs printed thereon. In some instances, consumers may prefer diapers with designs that extend contiguously over large areas of the diaper pants. Thus, in some configurations, diaper pants may include printed graphics that appear to extend contiguously across assembled components. In making such diapers, continuous substrates of material having printed graphics may be converted into different components that are combined to create the diapers. During the assembly process, the substrates of material having the graphics printed thereon may be subjected to various process transformations, such as folding, bonding, trimming, and/or cutting, before being combined to create the absorbent articles. However, subjecting printed substrates to various process transformations and/or combining operations with other printed components in areas where the graphics are located may create challenges in performing such process transformations when attempting to maintain aesthetically pleasing final assemblies. In addition, printing such graphics can add to the cost and complexity of the assembly process.

Consequently, there remains a need for absorbent articles with colors that appear to extend contiguously across some assembled components and/or provide stark contrast in colors of some assembled components without the need to print such components.

### SUMMARY

In one form, an absorbent article comprises: a first belt comprising a belt proximal edge and a belt distal edge longitudinally outboard of the proximal edge, and a first plurality of elastic strands positioned between and connected with a first nonwoven substrate and a second nonwoven substrate; a second belt, wherein laterally opposing end portions of the second belt are connected with laterally opposing end portions of the first belt at a first side seam and a second side seam; a chassis comprising a topsheet, a backsheet, and an absorbent core positioned between the topsheet and the backsheet, the chassis comprising a longitudinally extending first side edge and a longitudinally extending second side edge laterally separated from the first side edge by a first end edge and a second end edge longitudinally separate from the first end edge, and wherein longitudinally opposing end regions of the chassis are connected with the first belt and the second belt; wherein a portion of the backsheet overlaps an inner wearer facing surface of the first belt to define a chassis overlap region, and wherein the first belt comprises a first extensible region extending laterally between the chassis overlap region and the first side seam and a second extensible region extending laterally between the chassis overlap region and the second side seam; a waistband comprising a waistband distal edge and a waistband proximal edge, and a second plurality of elastic strands connected with a third nonwoven substrate, wherein a wearer facing surface of the waistband is connected with a garment facing surface of the first belt, wherein the waistband distal edge is positioned longitudinally outboard of the belt distal edge, and wherein the waistband proximal edge is positioned longitudinally inboard of the belt distal edge; a first color zone comprising a first color, the first color zone being defined by a garment facing surface of the waistband, and wherein the first color is defined by colorants in the third nonwoven substrate; and a second color zone comprising a second color, the second color zone being defined by the garment facing surface of the first belt in the chassis overlap region, and wherein the second color is defined by colorants in the first nonwoven substrate, the second nonwoven substrate, and the backsheet; and wherein the first color and the second color are different and comprise a delta E of at least 10, measured according to a Color Measurement Method.

### BRIEF DESCRIPTION OF THE DRAWINGS

FIG. 1 shows a perspective views of a diaper pant in a pre-fastened configuration.

FIG. 2A shows a plan view of a diaper pant with the portion of the diaper that faces away from a wearer oriented toward the viewer.

FIG. 2B shows a plan view of a diaper pant with the portion of the diaper that faces toward a wearer oriented toward the viewer.

FIG. 3 is a cross-sectional view of the diaper pant of FIG. 2A taken along line 3-3 showing first and second elastic belts as well as first and second waistbands.

FIG. 3A is a detailed cross-sectional view of a waistband from FIG. 3 configured as a laminate comprising a folded waistband substrate.

FIG. 3B is a detailed cross-sectional view of another configuration of a waistband configured as a laminate comprising a folded waistband substrate.

FIG. 3C is a detailed cross-sectional view of another configuration of a waistband configured as a laminate comprising a folded waistband substrate.

FIG. 3D is a detailed cross-sectional view of another configuration of a waistband configured as a laminate comprising a folded waistband substrate.

FIG. 4 is a cross-sectional view of a diaper pant including first and second elastic belts with a first substrate connecting the first and second elastic belts.

FIG. 5 shows a perspective view of a diaper pant in a pre-fastened configuration illustrating rugosities and components assembled with components constructed with substrate layers comprising colorants that may be combined to achieve desired color contrasts.

FIG. 6 is a front plan view of the diaper pant from FIG. 5 in a laid out flat configuration illustrating various color zones.

### DETAILED DESCRIPTION

The following term explanations may be useful in understanding the present disclosure:

“Absorbent article” is used herein to refer to consumer products whose primary function is to absorb and retain soils and wastes. Absorbent articles can comprise sanitary napkins, tampons, panty liners, interlabial devices, wound dressings, wipes, disposable diapers including taped diapers and diaper pants, inserts for diapers with a reusable outer cover, adult incontinent diapers, adult incontinent pads, and adult incontinent pants. The term “disposable” is used herein to describe absorbent articles which generally are not intended to be laundered or otherwise restored or reused as an absorbent article (e.g., they are intended to be discarded after a single use and may also be configured to be recycled, composted or otherwise disposed of in an environmentally compatible manner).

An “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

As used herein, the term “distal” is used to describe a position situated away from a center of a body or from a point of attachment, and the term “proximal” is used to describe a position situated nearer to a center of a body or a point of attachment.

The term “substrate” is used herein to describe a material which is primarily two-dimensional (i.e., in an XY plane) and whose thickness (in a Z direction) is relatively small (i.e., 1/10 or less) in comparison to its length (in an X direction) and width (in a Y direction). Non-limiting examples of substrates include a web, layer or layers or fibrous materials, nonwovens, films and foils such as polymeric films or metallic foils. These materials may be used alone or may comprise two or more layers laminated together. As such, a web is a substrate.

The term “nonwoven” refers herein to a material made from continuous (long) filaments (fibers) and/or discontinuous (short) filaments (fibers) by processes such as spunbonding, meltblowing, carding, and the like. Nonwovens do not have a woven or knitted filament pattern.

The term “machine direction” (MD) is used herein to refer to the direction of material flow through a process. In addition, relative placement and movement of material can be described as flowing in the machine direction through a process from upstream in the process to downstream in the process.

The term “cross direction” (CD) is used herein to refer to a direction that is generally perpendicular to the machine direction.

“Pre-strain” refers to the strain imposed on an elastic or elastomeric material prior to combining it with another element of the elastomeric laminate or the absorbent article. Pre-strain is determined by the following equation Pre-strain=((extended length of the elastic-relaxed length of the elastic)/relaxed length of the elastic)*100.

“Decitex” also known as Dtex is a measurement used in the textile industry used for measuring yarns or filaments. 1 Decitex=1 gram per 10,000 meters. In other words, if 10,000 linear meters of a yarn or filament weights 500 grams that yarn or filament would have a decitex of 500.

The term “taped diaper” (also referred to as “open diaper”) refers to disposable absorbent articles having an initial front waist region and an initial back waist region that are not fastened, pre-fastened, or connected to each other as packaged, prior to being applied to the wearer. A taped diaper may be folded about the lateral centerline with the interior of one waist region in surface to surface contact with the interior of the opposing waist region without fastening or joining the waist regions together. Example taped diapers are disclosed in various suitable configurations U.S. Pat. Nos. 5,167,897, 5,360,420, 5,599,335, 5,643,588, 5,674,216, 5,702,551, 5,968,025, 6,107,537, 6,118,041, 6,153,209, 6,410,129, 6,426,444, 6,586,652, 6,627,787, 6,617,016, 6,825,393, and 6,861,571; and U.S. Patent Publication Nos. 2013/0072887 A1; 2013/0211356 A1; and 2013/0306226 A1, all of which are incorporated by reference herein.

The term “pant” (also referred to as “training pant”, “pre-closed diaper”, “diaper pant”, “pant diaper”, and “pull-on diaper”) refers herein to disposable absorbent articles having a continuous perimeter waist opening and continuous perimeter leg openings designed for infant or adult wearers. A pant can be configured with a continuous or closed waist opening and at least one continuous, closed, leg opening prior to the article being applied to the wearer. A pant can be preformed or pre-fastened by various techniques including, but not limited to, joining together portions of the article using any refastenable and/or permanent closure member (e.g., seams, heat bonds, pressure welds, adhesives, cohesive bonds, mechanical fasteners, etc.). A pant can be preformed anywhere along the circumference of the article in the waist region (e.g., side fastened or seamed, front waist fastened or seamed, rear waist fastened or seamed). Example diaper pants in various configurations are disclosed in U.S. Pat. Nos. 4,940,464; 5,092,861; 5,246,433; 5,569,234; 5,897,545; 5,957,908; 6,120,487; 6,120,489; 7,569,039 and U.S. Patent Publication Nos. 2003/0233082 A1; 2005/0107764 A1, 2012/0061016 A1, 2012/0061015 A1; 2013/0255861 A1; 2013/0255862 A1; 2013/0255863 A1; 2013/0255864 A1; and 2013/0255865 A1, all of which are incorporated by reference herein.

“Body-facing” and “garment-facing” refer respectively to the relative location of an element or a surface of an element or group of elements. “Body-facing” implies the element or surface is nearer to the wearer during wear than some other element or surface. “Garment-facing” implies the element or surface is more remote from the wearer during wear than some other element or surface (i.e., element or surface is proximate to the wearer's garments that may be worn over the disposable absorbent article).

The present disclosure relates to absorbent articles including absorbent chassis, belts, and waistbands, and more particularly, to diaper pants comprising solid colored nonwoven webs and/or films combined so as to provide relatively stark color contrasts between waistbands and belts and relatively subtle color contrasts between absorbent chassis and belts. In some configurations, an absorbent article may include a first belt and a second belt, wherein laterally opposing end portions of the second belt are connected with laterally opposing end portions of the first belt at a first side seam and a second side seam. The first belt may comprise a belt proximal edge and a belt distal edge longitudinally outboard of the proximal edge, and a first plurality of elastic strands positioned between and connected with a first nonwoven substrate and a second nonwoven substrate. The absorbent article may also comprise a chassis comprising a topsheet, a backsheet, and an absorbent core positioned between the topsheet and the backsheet. The chassis may comprise a longitudinally extending first side edge and a longitudinally extending second side edge laterally separated from the first side edge by a first end edge and a second end edge longitudinally separate from the first end edge. Longitudinally opposing end regions of the chassis are connected with the first belt and the second belt. A portion of the backsheet may overlap an inner wearer facing surface of the first belt to define a chassis overlap region, and the first belt may comprise a first extensible region extending laterally between the chassis overlap region and the first side seam and a second extensible region extending laterally between the chassis overlap region and the second side seam. The absorbent article may also comprise a waistband comprising a waistband distal edge and a waistband proximal edge, and a second plurality of elastic strands connected with a third nonwoven substrate. A wearer facing surface of the waistband may be connected with a garment facing surface of the first belt. As such, the waistband distal edge may be positioned longitudinally outboard of the belt distal edge, and the waistband proximal edge may be positioned longitudinally inboard of the belt distal edge.

As discussed in more detail below, the absorbent article may comprise various color zones comprising colors defined by colorants in the components in the various color zones, wherein the colors comprise various delta E values depending on locations of the color zones relative to each other. For example, the absorbent article may comprise a first color zone and a second color zone. The first color zone may be defined by a garment facing surface of the waistband and may comprise a first color defined by colorants in the third nonwoven substrate. The second color zone may be defined by the garment facing surface of the first belt in the chassis overlap region and may comprise a second color defined by colorants in the first nonwoven substrate, the second nonwoven substrate, and the backsheet. In some configurations, the first color and the second color are different and comprise a delta E of at least 10, measured according to a Color Measurement Method. In some configurations, the first color and the second color may comprise a delta E of at least 13, measured according to the Color Measurement Method.

In some configurations, the absorbent article may also comprise a third color zone defined by the garment facing surface of the first belt in the first extensible region. The third color zone may comprise a third relaxed color when the first belt is in a fully relaxed state in the first extensible region. Absorbent articles herein may also comprise a fourth color zone defined by a garment facing surface of the backsheet extending longitudinally between the first belt and the second belt. The fourth color zone may comprise a fourth color. In some configurations, the fourth color and the third relaxed color comprise a delta E of less than 2, measured according to the Color Measurement Method. In some configurations, the fourth color and the third relaxed color may comprise a delta E of from 0 to about 3, measured according to the Color Measurement Method, specifically reciting all 0.1 delta E increments within the above-recited range and all ranges formed therein or thereby.

It is to be appreciated that the substrates made with colorants and assembled to form laminates discussed herein may be used to construct various types of components used in the manufacture of different types of absorbent articles, such as diaper pants. To help provide additional context to the subsequent discussion of absorbent articles comprising various color zones comprising colors defined by colorants within the substrates in the various color zones, the following provides a general description of absorbent articles in the form of diaper pants that include components and color zones that may be assembled with the substrates and laminates disclosed herein.

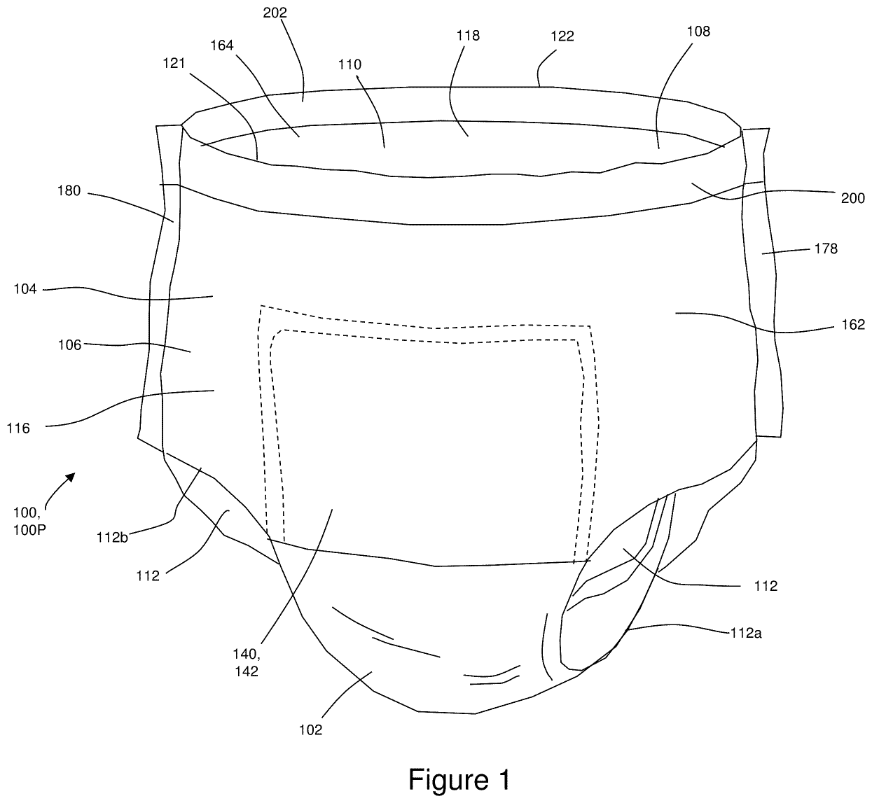

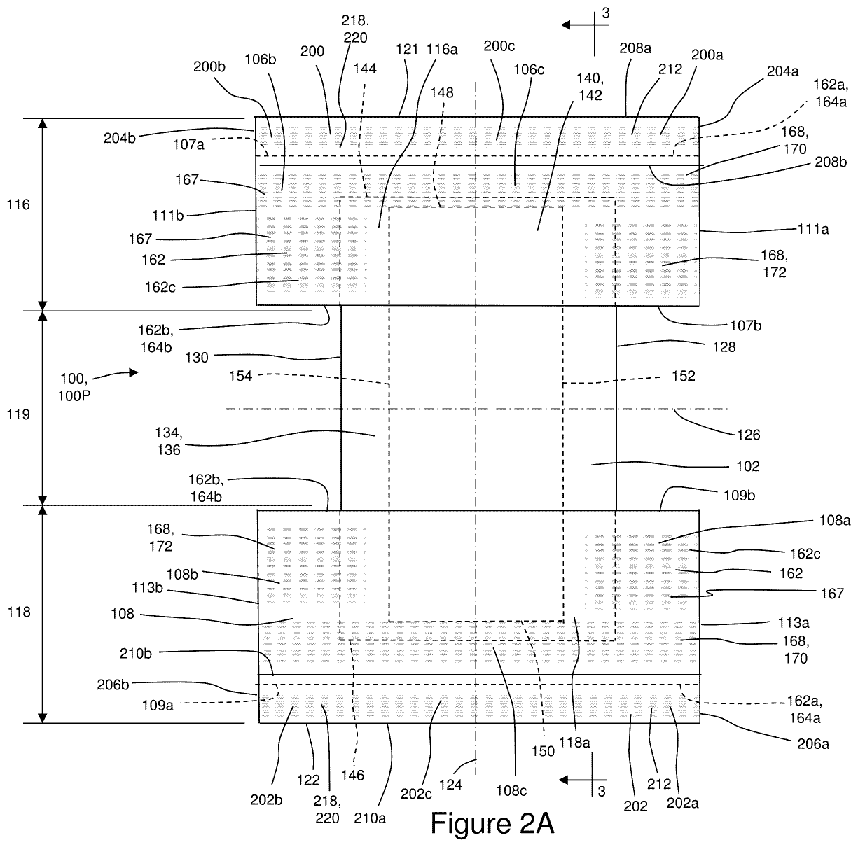

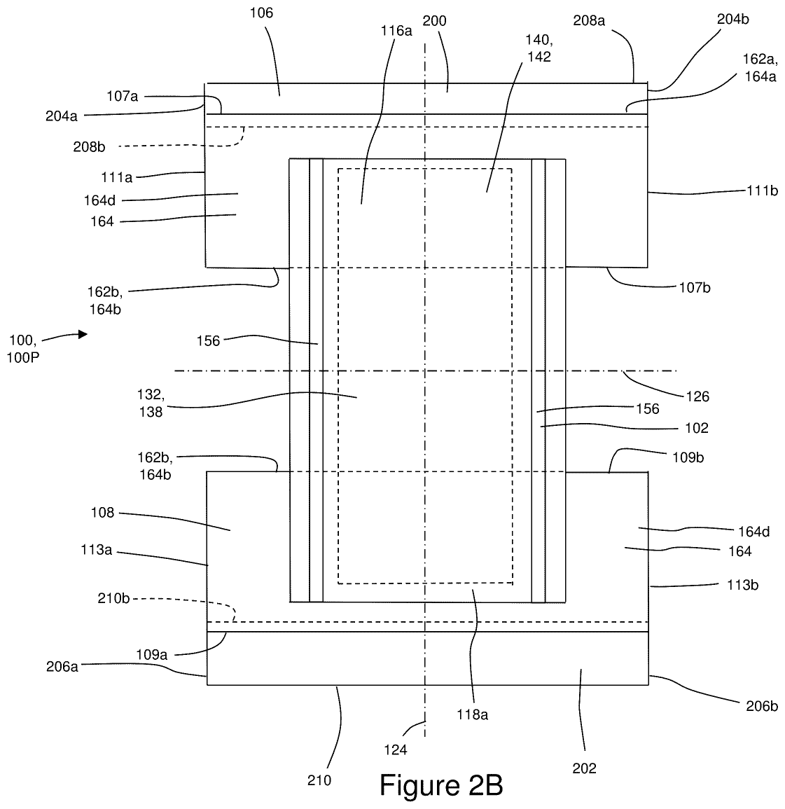

For the purposes of a specific illustration, FIGS. 1-2B show an example of an absorbent article 100 in the form of a diaper pant 100P that may include components constructed from substrates and laminates assembled to define the color zone configurations disclosed herein. In particular, FIG. 1 shows a perspective view of a diaper pant 100P in a pre-fastened configuration. FIG. 2A shows a plan view of the diaper pant 100P with the portion of the diaper that faces away from a wearer oriented toward the viewer, and FIG. 2B shows a plan view of the diaper pant 100P with the portion of the diaper that faces toward a wearer oriented toward the viewer. The diaper pant 100P includes a chassis 102, a first elastic belt 106, and a second elastic belt 108. As discussed in more detail below, the diaper pant 100P may also include a first elastic waistband 200 connected with the first elastic belt 106 and/or a second elastic waistband 202 connected with the second elastic belt 108. Opposing end regions of the first elastic belt 106, the second elastic belt 108, and one of or both of the first waistband 200 and the second waistband 202 may be connected together to form a ring-like belt 104.

With continued reference to FIGS. 1-2B, the diaper pant 100P and the chassis 102 each include a first waist region 116, a second waist region 118, and a crotch region 119 disposed intermediate the first and second waist regions. It may also be described that the chassis 102 includes a first end region 116a, a second end region 118a, and a crotch region 119 disposed intermediate the first and second end regions 116a, 118a. The first waist region 116 may be configured as a front waist region, and the second waist region 118 may be configured as back waist region. The diaper 100P may also include a laterally extending front waist edge 121 in the front waist region 116 and a longitudinally opposing and laterally extending back waist edge 122 in the back waist region 118. To provide a frame of reference for the present discussion, the diaper 100P and chassis 102 of FIGS. 2A and 2B are shown with a longitudinal axis 124 and a lateral axis 126. In some embodiments, the longitudinal axis 124 may extend through the front waist edge 121 and through the back waist edge 122. And the lateral axis 126 may extend through a first longitudinal or right side edge 128 and through a second longitudinal or left side edge 130 of the chassis 102.

As shown in FIGS. 1-2B, the diaper pant 100P may include an inner, body facing surface 132, and an outer, garment facing surface 134. The chassis 102 may include a backsheet 136 and a topsheet 138. The chassis 102 may also include an absorbent assembly 140, including an absorbent core 142, disposed between a portion of the topsheet 138 and the backsheet 136. As discussed in more detail below, the diaper 100P may also include other features, such as leg elastics and/or leg cuffs to enhance the fit around the legs of the wearer.

As shown in FIG. 2A, the periphery of the chassis 102 may be defined by the first longitudinal side edge 128, a second longitudinal side edge 130, a first laterally extending end edge 144 disposed in the first waist region 116, and a second laterally extending end edge 146 disposed in the second waist region 118. Both side edges 128 and 130 extend longitudinally between the first end edge 144 and the second end edge 146. As shown in FIG. 2A, the laterally extending end edges 144 and 146 are located longitudinally inward from the laterally extending front waist edge 121 in the front waist region 116 and the laterally extending back waist edge 122 in the back waist region 118. When the diaper pant 100P is worn on the lower torso of a wearer, the front waist edge 121 and the back waist edge 122 may encircle a portion of the waist of the wearer. At the same time, the side edges 128 and 130 may encircle at least a portion of the legs of the wearer. And the crotch region 119 may be generally positioned between the legs of the wearer with the absorbent core 142 extending from the front waist region 116 through the crotch region 119 to the back waist region 118.

As previously mentioned, the diaper pant 100P may include a backsheet 136. The backsheet 136 may also define a portion of the outer, garment facing surface 134 of the chassis 102. The backsheet 136 is generally that portion of the absorbent article 100 positioned proximate to the garment-facing surface of the absorbent assembly 140. The backsheet 136 may be joined to portions of the topsheet 138, the absorbent assembly 140, and/or any other layers of the absorbent article by any attachment methods known to those of skill in the art. The backsheet 136 prevents, or at least inhibits, the bodily exudates absorbed and contained in the absorbent core 142 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials, such as films, which permit vapors to escape from the absorbent article, while still preventing, or at least inhibiting, bodily exudates from passing through the backsheet. The backsheet 136 may also comprise a woven or nonwoven material, polymeric films such as thermoplastic films of polyethylene or polypropylene, and/or a multi-layer or composite materials comprising a film and a nonwoven material, such as having an inner film layer and an outer nonwoven layer or outer cover material. The outer cover material (sometimes referred to as a backsheet nonwoven) may comprise one or more nonwoven materials joined to a film. The outer cover material may form at least a portion of the garment facing surface 134 of the backsheet 136 so that film is not present on the garment facing surface 134. The outer cover material may comprise a bond pattern, apertures, and/or three-dimensional features. The outer cover material may be a hydroentangled nonwoven material or a variable basis weight nonwoven material. Such a material may have one or more layers.

Also described above, the diaper 100 may include a topsheet 138. The topsheet 138 may also define all or part of the inner, wearer facing surface 132 of the chassis 102. The topsheet 138 may be compliant, soft feeling, and non-irritating to the wearer's skin. It may be elastically stretchable in one or two directions. Further, the topsheet 138 may be liquid pervious, permitting liquids (e.g., menses, urine, and/or runny feces) to penetrate through its thickness. A topsheet 138 may be manufactured from a wide range of materials such as woven and nonwoven materials; apertured or hydroformed thermoplastic films; apertured nonwovens, porous foams; reticulated foams; reticulated thermoplastic films; and thermoplastic scrims. Woven and nonwoven materials may comprise natural fibers such as wood or cotton fibers; synthetic fibers such as polyester, polypropylene, or polyethylene fibers; or combinations thereof. If the topsheet 138 includes fibers, the fibers may be spunbond, carded, wet-laid, meltblown, hydroentangled, or otherwise processed as is known in the art.

Topsheets 138 may be selected from high loft nonwoven topsheets, apertured film topsheets and apertured nonwoven topsheets. Apertured film topsheets may be pervious to bodily exudates, yet substantially non-absorbent, and have a reduced tendency to allow fluids to pass back through and rewet the wearer's skin. Exemplary apertured films may include those described in U.S. Pat. Nos. 5,628,097; 5,916,661; 6,545,197; and 6,107,539, all of which are incorporated by reference herein.

As mentioned above, the diaper 100 may also include an absorbent assembly 140 that is joined to the chassis 102. As shown in FIGS. 1A and 1B, the absorbent assembly 140 may have a laterally extending front edge 148 in the front waist region 116 and may have a longitudinally opposing and laterally extending back edge 150 in the back waist region 118. The absorbent assembly may have a longitudinally extending right side edge 152 and may have a laterally opposing and longitudinally extending left side edge 154, both absorbent assembly side edges 152 and 154 may extend longitudinally between the front edge 148 and the back edge 150. The absorbent assembly 140 may additionally include one or more absorbent cores 142 or absorbent core layers. The absorbent core 142 may be at least partially disposed between the topsheet 138 and the backsheet 136 and may be formed in various sizes and shapes that are compatible with the diaper. Exemplary absorbent structures for use as the absorbent core of the present disclosure are described in U.S. Pat. Nos. 4,610,678; 4,673,402; 4,888,231; and 4,834,735, all of which are incorporated by reference herein.

Some absorbent core embodiments may comprise fluid storage cores that contain reduced amounts of cellulosic airfelt material. For instance, such cores may comprise less than about 40%, 30%, 20%, 10%, 5%, or even 1% of cellulosic airfelt material. Such a core may comprise primarily absorbent gelling material in amounts of at least about 60%, 70%, 80%, 85%, 90%, 95%, or even about 100%, where the remainder of the core comprises a microfiber glue (if applicable). Such cores, microfiber glues, and absorbent gelling materials are described in U.S. Pat. Nos. 5,599,335; 5,562,646; 5,669,894; and 6,790,798 as well as U.S. Patent Publication Nos. 2004/0158212 A1 and 2004/0097895 A1, all of which are incorporated by reference herein.

In some configurations, the absorbent assembly 140 may include an acquisition system disposed between the topsheet 138 and a wearer facing side of the absorbent core 142. The acquisition system may be in direct contact with the absorbent core 142 and may comprise a single layer or multiple layers, such as an upper acquisition layer (also referred to herein as a first acquisition layer) facing towards the wearer's skin and a lower acquisition layer (also referred to herein as a second acquisition layer) facing the garment of the wearer. In some embodiments, the acquisition system may function to receive a surge of liquid, such as a gush of urine. As such, the acquisition system may serve as a temporary reservoir for liquid until the absorbent core 142 can absorb the liquid. Exemplary acquisition systems and associated manufacturing processes are described in U.S. Pat. Nos. 8,603,277 and 8,568,566; U.S. Patent Publication Nos. 2012/0316046 A1 and 2014/0163504 A1, all of which are hereby incorporated by reference herein.

As previously mentioned, the diaper 100P may also include elasticized leg cuffs 156. It is to be appreciated that the leg cuffs 156 can be and are sometimes also referred to as leg bands, side flaps, barrier cuffs, elastic cuffs or gasketing cuffs. The elasticized leg cuffs 156 may be configured in various ways to help reduce the leakage of body exudates in the leg regions. Example leg cuffs 156 may include those described in U.S. Pat. Nos. 3,860,003; 4,909,803; 4,695,278; 4,795,454; 4,704,115; 4,909,803; and U.S. Patent Publication No. 2009/0312730 A1, all of which are incorporated by reference herein.

As mentioned above, diaper pants may be manufactured with a ring-like belt 104 and provided to consumers in a configuration wherein the front waist region 116 and the back waist region 118 are connected to each other as packaged, prior to being applied to the wearer. As such, diaper pants may have a continuous perimeter waist opening 110 and continuous perimeter leg openings 112 such as shown in FIG. 1. The ring-like belt may be formed by joining the front waist region 116 and the back waist region 118 at a first side seam 178 at a second side seam 180 to define the ring-like belt 104 as well as the waist opening 110 and leg openings 112. It is to be appreciated that the front waist region 116 and the back waist region 118 may be joined with permanent side seams or with an openable and reclosable fastening system.

As previously mentioned, the ring-like belt 104 may defined at least in part by a first elastic belt 106 connected with a second elastic belt 108. As shown in FIGS. 2A and 2B, the first elastic belt 106 extends between a first longitudinal side edge 111a and a second longitudinal side edge 111b and defines first and second opposing end regions 106a, 106b and a central region 106c. And the second elastic 108 belt extends between a first longitudinal side edge 113a and a second longitudinal side edge 113b and defines first and second opposing end regions 108a, 108b and a central region 108c. The central region 106c of the first elastic belt is connected with the first waist region 116 or first end region 116a of the chassis 102, and the central region 108c of the second elastic belt 108 is connected with the second waist region 118 or second end region 118a of the chassis 102.

As shown in FIGS. 2A and 2B, the first elastic belt 106 comprises an outer laterally extending edge, also referred to as a first belt distal edge 107a, and an inner laterally extending edge, also referred to as a first belt proximal edge 107b. And the second elastic belt 108 comprises an outer laterally extending edge, also referred to as a second belt distal edge 109a, and an inner laterally extending edge, also referred to as a second belt proximal edge 109b. As such, a perimeter edge 112a of one leg opening may be defined by portions of the first belt proximal edge 107b, the second belt proximal edge 109b, and the first longitudinal or right side edge 128 of the chassis 102. And a perimeter edge 112b of the other leg opening may be defined by portions of the first belt proximal edge 107b, the second belt proximal edge 109b, and the second longitudinal or left side edge 130 of the chassis 102.

It is to be appreciated that the first elastic belt 106 and the second elastic belt 108 may define different sizes and shapes. For example, the first elastic belt 106 and the second elastic belt 108 may both define generally rectangular shapes. In some configurations, at least one of the first elastic belt 106 and the second elastic belt 108 may comprise lateral edges having different lengths. For example, the first elastic belt 106 may define a generally rectangular shape, and wherein the outer laterally extending edge 109a of the second elastic belt 108 and the inner laterally extending edge 109b may have different lengths. In some configurations, both the first elastic belt 106 and the second elastic belt 108 may comprise lateral edges having different lengths. For example, the outer laterally extending edge 107a of the first elastic belt 106 and the inner laterally extending edge 107b may have different lengths, and wherein the outer laterally extending edge 109a of the second elastic belt 108 and the inner laterally extending edge 109b may have different lengths. In some examples, the first elastic belt 106 may define a longitudinal length extending between outer laterally extending edge 107a and the inner laterally extending edge 107b, and the second elastic belt 108 may define a longitudinal length extending between outer laterally extending edge 109a and the inner laterally extending edge 109b. In some configurations, the longitudinal length of the first elastic belt 106 and the second elastic belt 108 may be the same or different.

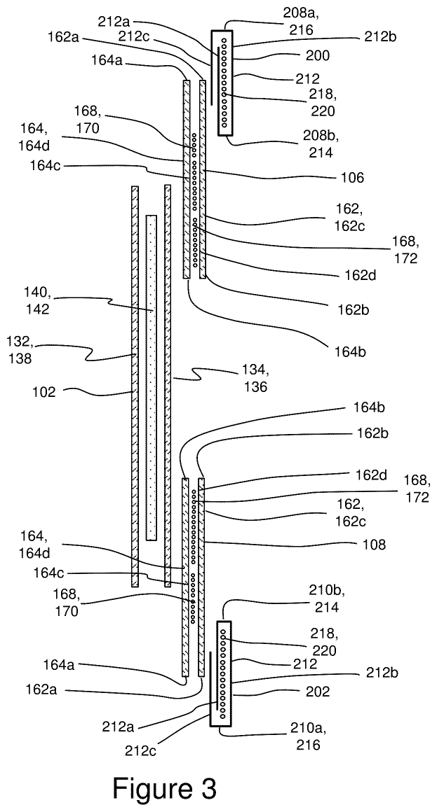

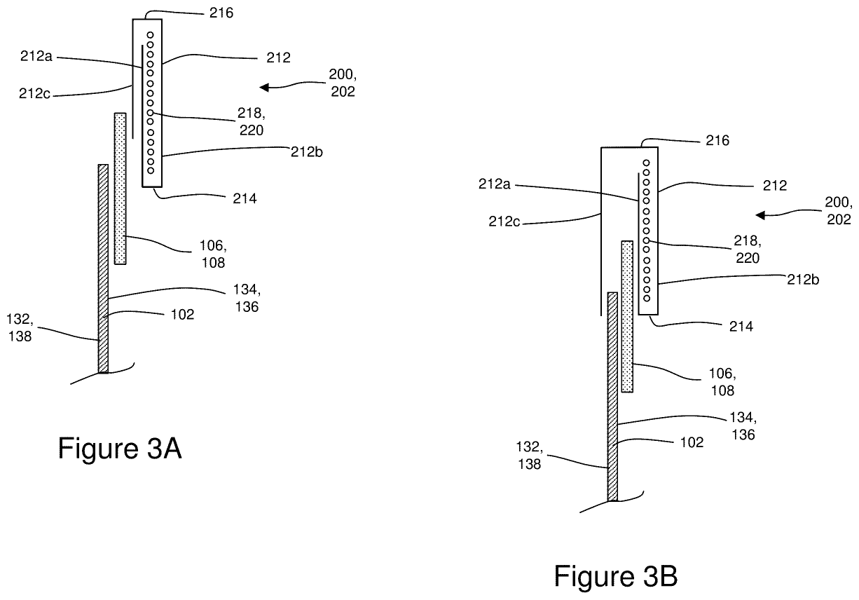

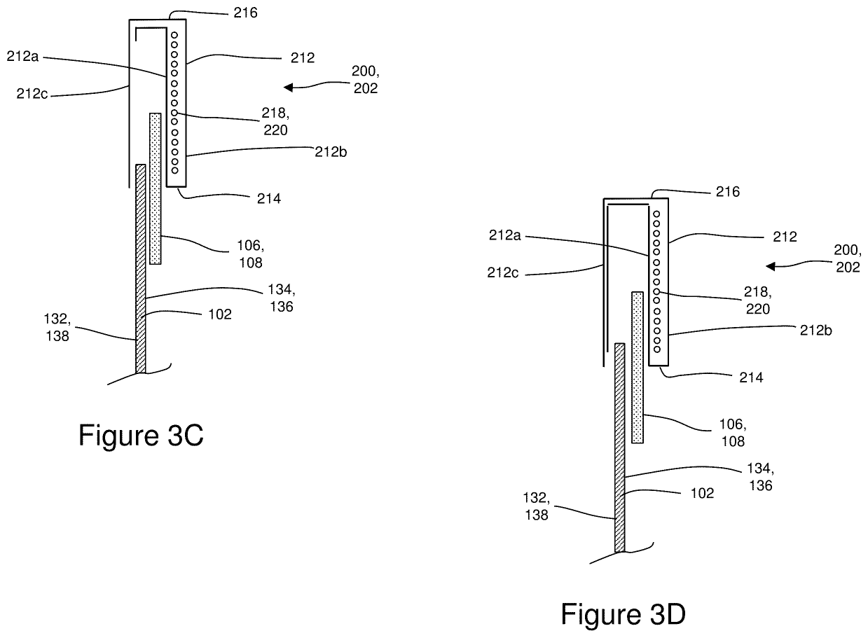

With reference to FIGS. 2A, 2B, and 3, the first elastic belt 106 and the second elastic belt 108 may also each comprise a first substrate 162 and a second substrate 164. The first substrates 162 may be oriented to define at least a portion of the garment facing surfaces of the first elastic belt 106 and the second elastic belt 108, and the second substrates 164 may be oriented to define at least a portion of the wearer facing surfaces of the first elastic belt 106 and the second elastic belt 108. The first substrate 162 may extend from a proximal edge 162b to a distal edge 162a, and the second substrate 164 may extend from a proximal edge 164b to a distal edge 164a. It is to be appreciated that the distal edge 162a and/or the proximal edge 162b of the first substrate 162 may be straight and/or curved and/or may be parallel or unparallel to each other. It is also to be appreciated that the distal edge 164a and/or the proximal edge 164b of the second substrate 164 may be straight and/or curved and/or may be parallel or unparallel to each other. In some configurations, the distal edge 162a of the first substrate 162 may define at least a portion of the first belt distal edge 107a and/or at least a portion of the second belt distal edge 109a, and/or the distal edge 164a of the second substrate 164 may define at least a portion of the first belt distal edge 107a and/or at least a portion of the second belt distal edge 109a. It is also to be appreciated that the first substrate 162 and/or the second substrate 164 may extend continuously from the first belt 106 to the second belt 108.

In some configurations, the proximal edge 162b of the first substrate 162 and/or the proximal edge 164b of the second substrate 164 may extend laterally across the backsheet 134. As such, the first belt proximal edge 107b and/or the second belt proximal edge 109b may extend laterally across the backsheet 134. As shown in FIGS. 2A-3, the first substrate 162 includes a garment facing surface 162c and an opposing wearer facing surface 162d, and the second substrate 164 includes a garment facing surface 164c and an opposing wearer facing surface 164d.

Belt elastic material 167 may be positioned between the wearer facing surface 162d of the first substrate 162 and the garment facing surface 164c of the second substrate 164. It is to be appreciated that the belt elastic material 167 may include one or more elastic elements such as strands, ribbons, elastic films, or panels extending along the lengths of the elastic belts. As shown in FIGS. 2A and 3, the elastic material 167 may include a plurality of elastic strands 168. In some configurations, the elastic strands 168 may be referred to herein as outer, waist elastics 170 and inner, waist elastics 172. Elastic strands 168, such as the outer waist elastics 170, may continuously extend laterally between the first and second opposing end regions 106a, 106b of the first elastic belt 106 and between the first and second opposing end regions 108a, 108b of the second elastic belt 108. In some embodiments, some elastic strands 168, such as the inner waist elastics 172, may be configured with discontinuities in areas, such as for example, where the first and second elastic belts 106, 108 overlap portions of the chassis 102, such as the backsheet 134 and/or the absorbent assembly 140. In some configurations, the first elastic belt 106 and/or second elastic belt 108 may define curved contours. For example, the inner lateral edges 107b, 109b of the first and/or second elastic belts 106, 108 may include non-linear or curved portions in the first and second opposing end regions. Such curved contours may help define desired shapes to leg opening 112, such as for example, relatively rounded leg openings. In addition to having curved contours, the elastic belts 106, 108 may include elastic strands 168 that extend along non-linear or curved paths that may correspond with the curved contours of the inner lateral edges 107b, 109b.

As shown in FIGS. 2A-3, the diaper pant 100P may also include a first waistband 200 and/or a second waistband 202, which may comprise a substrate or a laminate of substrate layers. As shown in FIGS. 2A and 2B, the first waistband 200 extends between a first longitudinal side edge 204a and a second longitudinal side edge 204b and defines first and second opposing end regions 200a, 200b and a central region 200c. And the second waistband 202 extends between a first longitudinal side edge 206a and a second longitudinal side edge 206b and defines first and second opposing end regions 202a, 202b and a central region 202c. The waistband 200 comprises an outer laterally extending edge, also referred to as a first waistband distal edge 208a, and an inner laterally extending edge, also referred to as a first waistband proximal edge 208b. And the second waistband 202 comprises an outer laterally extending edge, also referred to as a second waistband distal edge 210a, and an inner laterally extending edge, also referred to as a second waistband proximal edge 210b.

It is to be appreciated that the first waistband distal edge 208a and/or the first waistband proximal edge 208b may be straight and/or curved and/or may be parallel or unparallel to each other. It is also to be appreciated that the second waistband distal edge 210a and/or the second waistband proximal edge 210b may be straight and/or curved and/or may be parallel or unparallel to each other.

As shown in FIGS. 2A, 2B, and 3, a wearer facing surface of the first waistband 200 may be connected with a garment facing surface of the first belt 106. As such, the first waistband proximal edge 208b may be positioned longitudinally inboard of the first belt distal edge 107a. The first waistband distal edge 208a may be positioned longitudinally outboard of the first belt distal edge 107a. In some configurations, first waistband distal edge 208a may be coterminous with the first belt distal edge 107a. In addition, a wearer facing surface of the second waistband 202 may be connected with a garment facing surface of the second belt 108. As such, the second waistband proximal edge 210b may be positioned longitudinally inboard of the second belt distal edge 109a. The second waistband distal edge 210b may be positioned longitudinally outboard of the second belt distal edge 109a. In some configurations, the second waistband distal edge 210a may be coterminous with the second belt distal edge 109a. Thus, it is to be appreciated that, the first belt distal edge 107a and/or the first waistband distal edge 208a may define the front waist edge 121 of the diaper pant 100P, and the second belt distal edge 109a and/or the second waistband distal edge 210a. In some configurations, the first belt distal edge 107a, the second belt distal edge 109a, the first waistband distal edge 208a, and/or the second waistband distal edge 210a may define at least a portion of the waist opening 110.

As shown in FIG. 1, the first end region 106a of the first elastic belt 106 is connected with the first end region 108a of the second elastic belt 108 at first side seam 178, and the second end region 106b of the first elastic belt 106 is connected with the second end region 108b of the second elastic belt 108 at second side seam 180. In addition, the first end region 200a of the first waistband 200 may be connected with the first end region 202a of the second waistband 108 at first side seam 178, and the second end region 200b of the first waistband 200 may be connected with the second end region 202b of the second waistband 202 at second side seam 180.

It is to be appreciated that the diaper pant 100P may include only one of the first waist band 200 and the second waistband 202. As such, in some configurations, the first end region 200a of the first waistband 200 may be connected with the first end region 108a of the second elastic belt 108 at first side seam 178, and the second end region 200b of the first waistband 200 may be connected with the second end region 108b of the second elastic belt 108 at second side seam 180. In some configurations, the first end region 202a of the second waistband 202 may be connected with the first end region 106a of the first elastic belt 106 at first side seam 178, and the second end region 202b of the second waistband 202 may be connected with the second end region 106b of the first elastic belt 106 at second side seam 180.

It is to be appreciated that the first and/or second waistband 200, 202 may be configured as a single layer substrate or a laminate comprising two or more layers. It is to be appreciated that such layers may be defined by discrete substrates and/or portions of a single substrate. For example, as shown in FIGS. 3 and 3A, the first waistband 200 and/or the second waistband 202 may comprise a waistband substrate 212 folded onto itself to form a laminate. As shown in FIGS. 3 and 3A, the waistband substrate 212 may comprise a first fold line 214, wherein the waistband substrate 212 may comprise a first portion 212a and a second portion 212b extending from the first fold line 214, and the first portion 212a may be in a facing relationship with the second portion 212b. The waistband substrate 212 may also comprise a second fold line 216, wherein the second portion 212b and a third portion 212c extend from the second fold line 216. The third portion 212c may be positioned in a facing relationship with the first portion 212a, and the first portion 212a may be positioned or sandwiched between the second portion 212b and the third portion 212c. As such, the second portion 212b may extend between the first fold line 214 and the second fold line 216.

In some configurations, the first portion 212a may extend completely from the first fold line 214 to the second fold line 216. In some configurations, the first portion 212a may not extend completely from the first fold line 214 to the second fold line 216, and as such, some of the third portion 212c may be in a facing relationship with some of the second portion 212b. In some configurations, the first fold line 214 may define the proximal edge 208b, 210b of the first waistband 200 or the second waistband 202, respectively. In some configurations, the second fold line 216 may define the distal edge 208a, 210a of the first waistband 200 or the second waistband 202, respectively.

In some configurations, the second portion 212b may be oriented to define a garment facing surface of the first waistband 200 and/or the second waistband 202. In some configurations, the third portion 212c may extend completely from the second fold line 216 to the the first fold line 214 or beyond the first fold line. As such, in some configurations, the third portion 212c may be oriented to define a wearer facing surface of the first waistband 200 and/or the second waistband 202. In some configurations, the third portion 212c may not extend completely from the second fold line 216 to the first fold line 214, and as such, some of the third portion 212c and some of the first portion 212a may define the wearer facing surface of the first waistband 200 and/or the second waistband 202.

In another waistband configuration shown in FIG. 3B, the third portion 212c of the waistband substrate 212 may be configured to extend from the second fold 216 and over the belt 106, 108 and may be bonded with the topsheet 138 of the chassis 102. In turn, the first portion 212a of the waistband substrate 212 may bonded with the backsheet 134 of the chassis 102. In yet another waistband configuration shown in FIG. 3C, the first portion 212a of the waistband substrate 212 may extend into the second fold 216. In still another waistband configuration shown in FIG. 3D, the first portion 212a of the waistband substrate 212 may extend from the second fold 216 and over the belt 106, 108 and may be bonded with the topsheet 138 of the chassis 102. As such, the first portion 212a of the waistband substrate 212 may be bonded and sandwiched between the third portion 212c and the topsheet 138.

As shown in FIGS. 2A and 3, the first waistband 200 and/or the second waistband 202 may comprise waistband elastic material 218. It is to be appreciated that the waistband elastic material 218 may include one or more elastic elements such as strands, ribbons, elastic films, or panels extending along the lengths of the waistbands. For example, the waistband elastic material 218 may comprise a plurality of elastic strands 220 connected with the waistband substrate 212. In some configurations, elastic strands 220 may be positioned between the first portion 212a and the second portion 212b of the waistband substrate 212. In some configurations, elastic strands may be positioned between the third portion 212c and the second portion 212b of the waistband substrate 212.

It is to be appreciated that the first belt 106, the second belt 108, the first waistband 200, and the second waistband 202 may be configured in various different ways and may be constructed from various different materials. In some configurations, the first belt 106, the second belt 108, the first waistband 200, and/or the second waistband 202 may comprise the same materials and/or may have the same structure. In some embodiments, the first belt 106, the second belt 108, the first waistband 200, and/or the second waistband 202 may comprise different materials and/or may have different structures. It should also be appreciated that components of the first belt 106, the second belt 108, the first waistband 200, and/or the second waistband 202, such as the first substrate 162, second substrate 164, and/or waistband substrate 212, may be constructed from various materials. For example, the first substrate 162, the second substrate 164, and/or waistband substrate 212 that may be manufactured from materials such as plastic films; apertured plastic films; woven or nonwoven webs of natural materials (e.g., wood or cotton fibers), synthetic fibers (e.g., polyolefins, polyamides, polyester, polyethylene, or polypropylene fibers) or a combination of natural and/or synthetic fibers; or coated woven or nonwoven webs. In some configurations, the first substrate 162, the second substrate 164, and/or waistband substrate 212 may comprise a nonwoven web of synthetic fibers, and may include a stretchable nonwoven. In some configurations, the first belt 106, the second belt 108, the first waistband 200, and/or the second waistband 202 may include an inner hydrophobic, non-stretchable nonwoven material and an outer hydrophobic, non-stretchable nonwoven material.

It is to be appreciated that the first elastic belt 106, the second elastic belt 108, the first waistband 200, and/or the second waistband 202 may include various configurations of belt elastic materials 167 and waistband elastic materials 218 arranged in relation to each other and to the first substrate 162, second substrate 164, and/or the waistband substrate 212, such as disclosed for example in U.S. Patent Publication No. 2022/0142828 A1, which is incorporated by reference herein. As discussed above, the belt elastic material 167 and the waistband elastic material 218 may include configurations of one or more elastic elements such as strands, ribbons, films, or panels positioned in various arrangements. In some configurations, the belt elastic material 167 and/or the waistband elastic material 218 may comprise various elastics, elastic features and arrangements, and processes for assembly to create various elastomeric laminates, such as described in U.S. Patent Publication Nos. 2018/0168878 A1; 2018/0168877 A1; 2018/0168880 A1; 2018/0170027 A1; 2018/0169964 A1; 2018/0168879 A1; 2018/0170026 A1; 2018/0168889 A1; 2018/0168874 A1; 2018/0168875 A1; 2018/0168890 A1; 2018/0168887 A1; 2018/0168892 A1; 2018/0168876 A1; 2018/0168891 A1; 2019/0070042 A1; 2019/0070041 A1; 20210275362 A1; and 2022/0142828 A1 and combinations thereof, all of which are incorporated herein by reference. It is also to be appreciated the elastic materials 167, 218 herein may be configured with identical or different colors in various different locations on the first elastic belt 106, the second elastic belt 108, the first waistband 200, and/or the second waistband 202.

In some configurations, the belt elastic strands 168 and/or the waistband elastic strands 220 may be disposed at a constant interval in the longitudinal direction. In other configurations, the belt elastic strands 168 and/or the waistband elastic strands 220 may be disposed at different intervals in the longitudinal direction. In some configurations, the belt elastic material 167 and/or the waistband elastic material 218 in a stretched condition may be interposed and joined between uncontracted substrate layers. When the elastic material 167, 218 is relaxed, the elastic material 167, 218 returns to an unstretched condition and contracts the substrate layers. The elastic materials 167, 218 may provide a desired variation of contraction force in the first elastic belt 106, the second elastic belt 108, the first waistband 200, and/or the second waistband 202. It is to be appreciated that the chassis 102 and elastic belts 106, 108 may be configured in different ways other than as depicted in attached Figures. It is also to be appreciated that the elastic material 167, 218 may be joined to substrates continuously or intermittently along the interface between the elastic material 167, 218 and the substrates.

It is also to be appreciated that the first elastic belt 106 and/or the second elastic belt 108, the first waistband 200, and/or the second waistband 202 may be configured to include various quantities of belt elastic strands 168 and waistband elastic strands 220, respectively. In some configurations, the first elastic belt 106 and/or the second elastic belt 108, the first waistband 200, and/or the second waistband 202 may comprise from about 10 to about 1500 belt elastic strands 168 or waistband elastic strands 220. It is also to be appreciated that belt elastic strands 168 and waistband elastic strands 220 herein may comprise various Dtex values, strand spacing values, and pre-strain values and such elastic strands be may utilized with other elastic strands to create first and second elastic belts 106, 108 and/or first and second waistbands 200, 202 comprising elastic strands in various combinations of Dtex values, strand spacing values, and pre-strain values. For example, in some configurations, the Dtex of one or more belt elastic strands 168 and/to waistband elastic strands 220 may be greater than 500. In some configurations, the Dtex of one or more belt elastic strands 168 and/or waistband elastic strands 220 may be from about 10 to about 500, specifically reciting all 1 Dtex increments within the above-recited range and all ranges formed therein or thereby. In some configurations, a plurality of belt elastic strands 168 and/or waistband elastic strands 220 may be separated from each other by less than or equal to 4 mm. In some configurations, a plurality of belt elastic strands 168 and/or waistband elastic strands 220 may be separated from each other by about 0.25 mm to about 4 mm, specifically reciting all 0.01 mm increments within the above-recited range and all ranges formed therein or thereby. In some configurations, a plurality of belt elastic strands 168 and/or waistband elastic strands 220 may be separated from each other by greater than 4 mm. In some configurations, the pre-strain of the belt elastic strands 168 and/or waistband elastic strands 220 may be from about 50% to about 400%, specifically reciting all 1% increments within the above-recited range and all ranges formed therein or thereby. In some configurations, a plurality of belt elastic strands 168 and/or waistband elastic strands 220 may be in the form of extruded elastic strands, which may also be bonded with the substrates in a pre-corrugated configuration, such as disclosed for example in U.S. Pat. No. 5,681,302, which is incorporated by reference herein.

It is to be appreciated that the first elastic belt 106, the second elastic belt 108, the first waistband 200, and/or the second waistband 202 and components thereof may be bonded together in various ways. It is also to be appreciated that the first substrate 162, second substrate 164, waistband substrate 212, and/or elastic materials 167, 218 may be bonded together and/or with other components, such as the chassis 102, with adhesive and/or mechanical bonds. It is to be appreciated that adhesive and mechanical bonding methods may be utilized alone or in combination with each other.

In some configurations, adhesive may be applied to at least one of the first substrate 162, second substrate 164, waistband substrate 212, and/or elastic materials 167, 218 when being combined to form the first elastic belt 106, the second elastic belt 108, the first waistband 200, and/or the second waistband 202. Such adhesive may be applied by with adhesive applicator devices configured in various ways, such as for example, spray nozzles and/or slot coating devices.

In some configurations, the adhesive applicator devices may be configured in accordance with the apparatuses and/or methods disclosed in U.S. Pat. Nos. 8,186,296; 9,265,672; 9,248,054; and 9,295,590 and U.S. Patent Publication No. 2014/0148773 A1, all of which are incorporated by reference herein.

In some configurations, mechanical bonding devices may apply mechanical bonds to the to at least one of the first substrate 162, second substrate 164, waistband substrate 212, and/or elastic materials 167, 218 when being combined to form the first elastic belt 106, the second elastic belt 108, the first waistband 200, and/or the second waistband 202. Such mechanical bonds may be applied with heat, pressure, and/or ultrasonic devices. Examples of ultrasonic bonding devices, which may include linear or rotary type configurations, are disclosed for example in U.S. Pat. Nos. 3,113,225; 3,562,041; 3,733,238; 5,110,403; 6,036,796; 6,508,641; and 6,645,330. In some configurations, the ultrasonic bonding device may be configured as a linear oscillating type sonotrode, such as for example, available from Herrmann Ultrasonic, Inc. Examples of such mechanical bonding devices and methods are disclosed in U.S. Pat. Nos. 4,854,984; 6,291,039; 6,248,195; 8,778,127; and 9,005,392; and U.S. Patent Publication Nos. 2014/0377513 A1; and 2014/0377506 A1, all of which are incorporated by reference herein. In some configurations, mechanical bonding devices may apply bonds that bond the first substrate 162, second substrate 164, waistband substrate 212, and/or elastic materials 167, 218 together and/or may act to trap or immobilize discrete lengths of the contracted elastic strands.

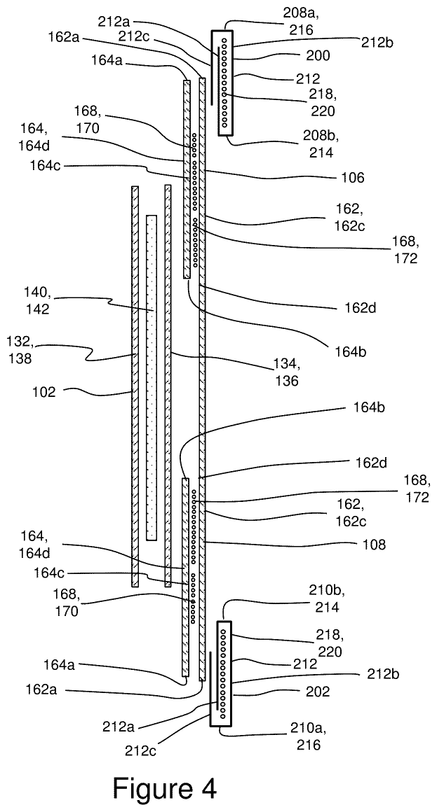

In addition to the various configurations of waistbands 200, 202 discussed herein, it is to be appreciated that the first elastic belt 106 and the second elastic belt 108 may be configured in various ways with various configurations and/or combinations of the first substrate 162 and the second substrate 164. In some configurations, the first substrate 162 and/or the second substrate 164 may be configured as single substrate layer or a laminate structure including a plurality of substrate layers. In yet another configuration, such as shown in FIG. 4, the first and second elastic belts 106, 108 may include a portion of a single garment facing layer of the first substrate 162 that forms part of the first elastic belt 106 and the second elastic belt 108 and extends longitudinally between the first elastic belt 106 and the second elastic belt 108 along the backsheet 136 of the chassis 102.

It is to be appreciated that the first elastic belt 106, the second elastic belt 108, the first waistband 200, and/or the second waistband 202 may be configured in various ways, such as disclosed for example in U.S. Patent Publication No. 2022/0142828 A1, which is incorporated by reference herein. For example, in some configurations, the first elastic belt 106, the second elastic belt 108, the first waistband 200, and/or the second waistband 202 may be configured with the same or different tensioning, rugosities, bonding patterns, and/or aperture arrangements. For example, along equal lengths in a longitudinal direction, the first waistband 200 and/or the second waistband 202 may be configured to apply greater tensile forces in a lateral direction than the first elastic belt 106 and/or the second elastic belt 108. In some configurations, the first elastic belt 106, the second elastic belt 108, the first waistband 200, and/or the second waistband 202 may comprise printed indicia and/or printed graphics comprising aesthetic images or designs and may convey various types of information to a user, such as for example, article size and/or front/back orientations of the absorbent article.

As mentioned above, absorbent articles may be configured with components combined in such a manner to provide relatively stark color contrasts between some components and subtle color contrasts between other components. For example, in the context of the discussion above, diaper pants 100P may comprise components such as waistbands 200, 202; belts 106, 108; and chassis 102 configured with relatively stark color contrasts between waistbands 200, 202 and belts 106, 108 and relatively subtle color contrasts between absorbent chassis 102 and belts 106, 108. Instead of printing such components, the waistbands 200, 202; belts 106, 108; and chassis 102 may comprise nonwoven substrates and/or film substrates comprising colorants to achieve the desired color contrasts.

In particular, nonwoven substrates and film substrates of the present disclosure may comprise a colorant which may be used in absorbent articles that are worn by consumers. Such nonwoven substrates and film substrates may be used to manufacture portions of an absorbent article, such as components comprising a laminate, that are consumer pleasing and premium looking. The term “colorant”, as used herein, refers to inks, dyes, pigments, or the like, and combinations thereof, used to create color in a substrate.

Generally, colorants, such as pigments, are added to the thermoplastic material in the form of a masterbatch or concentrate at the time of formation of the fibers. For nonwoven substrates that are formed via a spunmelt process, e.g., spunbond, meltblown, etc., polymeric material is extruded through a plurality of holes in a die. Therefore, the colorant must pass through the holes in the die along with the polymeric material. The colorant may thus be locked in the polymer matrix of the filament. The use of colorants in nonwoven substrates may provide certain advantages, such as preventing colorant from rubbing off on a wearer.

A colorant masterbatch may be added to the polymer formulation of spunbond and/or meltblown fibers at about 0.25 percent by weight to about 8 percent by weight, about 0.5 percent by weight to about 6 percent by weight, about 0.75 percent by weight to about 5 percent by weight, about 1 percent by weight to about 4.5 percent by weight, about 1.25 percent by weight to about 4 percent by weight, or about 1.5 percent by weight to about 3.5 percent by weight, specifically reciting all values within these ranges and any ranges created thereby.

Some colorants may comprise particles having sizes less than about 2 μm or less than about 1 μm. In some configurations, colorants may comprise particles having sizes ranging from about 8 nm to about 100 nm.

A pigment is a material, which can be organic or inorganic and may include activatable, structural and or special effects pigments. A pigment changes the color of reflected or transmitted light as the result of wavelength-selective absorption. This physical process differs from fluorescence, phosphorescence, and other forms of luminescence, in which a material emits light. A pigment is a generally insoluble powder, which differs from a dye, which either is itself a liquid or is soluble in a solvent (resulting in a solution). The pigment will typically be mixed with the thermoplastic material, of which the fibers are made. Often, the pigment is added to the thermoplastic material in the form of a masterbatch or concentrate at the time of formation of the fibers. Colored masterbatches useful for the present invention include polypropylene based custom color masterbatches e.g., supplied by Ampacet; Lufilen and Luprofil supplied by BASF; and Remafin for polyolefin fibers, Renol-AT for polyester fibers, Renol-AN for polyamide fibers and CESA for renewable polymers supplied by Clariant. Hence, the pigment will be suspended in the molten thermoplastic material prior to the thermoplastic material being forced through the spinnerets to form and lay down the fibers which form the nonwoven substrate.

To increase the whiteness and/or opacity of the fibers, TiO2 may be used. Different crystal forms are available, however most preferred are rutile or anatase TiO2. Other white pigments include zinc oxide, zinc sulfide, lead carbonate or calcium carbonate. To create a black color, carbon black or any other suitable colorant may be used. Various colored inorganic pigments may be used depending upon the desired color and may include metal oxides, hydroxides and sulfides or any other suitable material. Non-limiting examples of inorganic pigments include cadmium orange, iron oxide, ultramarine, chrome oxide green. One or more pigments may be combined to create the desired color. Non-limiting examples of organic colorants include anthraquinone pigments, azo pigments, benzimidazolone pigments, BONA Lakes, Dioxazine, Naphthol, Perylene, Perinone, Phthalocyanine, Pyranthrone, Quinacridones. Effects pigments including metal, pearlescent and fluorescent may also be used. Various colorants are described in Plastics Additives Handbook, 5th Edition.

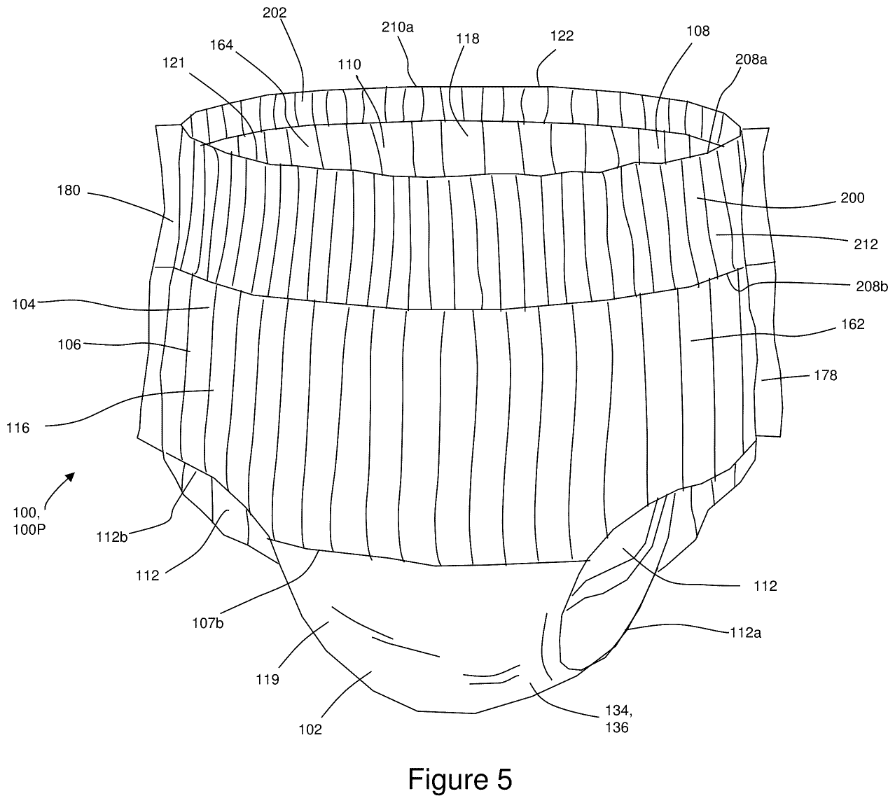

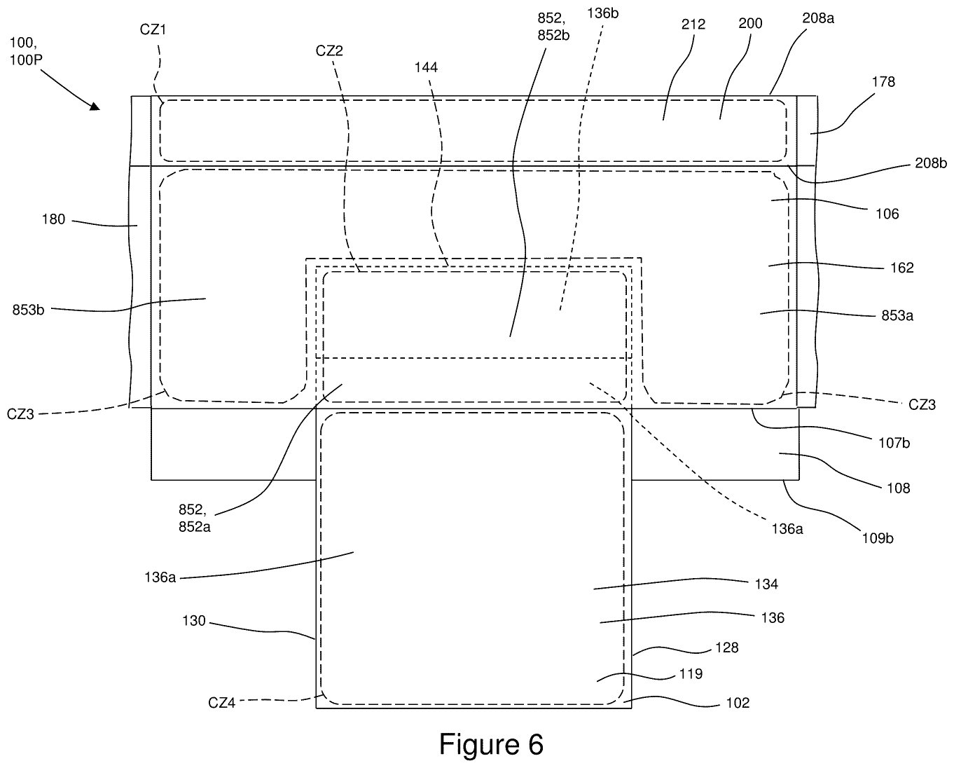

FIG. 5 shows a perspective view of an example diaper pant 100P in a pre-fastened configuration illustrating rugosities and components assembled with components constructed with substrate layers comprising colorants that may be combined to achieve desired color contrasts. FIG. 6 is a front plan view of the diaper pant 100P from FIG. 5 in a laid out flat configuration with various color zones defined on components of the diaper pant 100P. As shown in FIG. 6, a portion of the backsheet 136 overlaps an inner wearing facing surface of the first belt 106 to define a chassis overlap region 852.

The first belt 106 comprises a first extensible region 853a extending laterally between the chassis overlap region 852 and the first side seam 178 and a second extensible region 853b extending laterally between the chassis overlap region 852 and the second side seam 180. In some configurations, the backsheet 136 may comprise a laminate comprising a backsheet nonwoven substrate 136a bonded with a backsheet film substrate 136b. In some configurations, the backsheet nonwoven substrate 136a completely covers the backsheet film substrate 136b, such that the entirety of the garment facing surface of the backsheet 136 is defined by the backsheet nonwoven substrate 136a. In some configurations, the backsheet nonwoven substrate 136a and the backsheet nonwoven film 136b are different sizes. In some configurations, the backsheet film substrate 136b may be longer in the longitudinal direction than the backsheet nonwoven substrate 136a. As such, a portion of the garment facing surface of the backsheet 136 may be defined by the backsheet nonwoven substrate 136a and a portion of the garment facing surface of the backsheet 136 may be defined by the backsheet film substrate 136a. Thus, in some configurations, such as shown in FIG. 6, the chassis overlap region 852 may comprise a first area 852a wherein the backsheet nonwoven substrate 136a is bonded with the inner wearer facing surface of the first belt 106 and a second area 852b wherein the backsheet film substrate 136b is bonded with the inner wearer facing surface of the first belt 106. In some configurations, the second area 852b of the chassis overlap region 852 may be positioned longitudinally between the first end edge 144 of the chassis 102 and the first area 852a of the chassis overlap region 852. In some configurations, some or none of the elastic strands 168 of the first belt 106 may extend laterally across the second area 852b of the chassis overlap region 852, and some or none of the elastic strands 168 of the first belt may not extend across the first area 852a of the chassis overlap region 852. It is to be appreciated that structural features and color zones illustrated and described with reference to FIG. 6 relating to the first belt 106 and chassis 102 of the diaper pant 100P may also be applicable to the second belt 108 and chassis 102 of the diaper pant 100P.

With continued reference to FIG. 6, the diaper pant 100P may comprise various color zones, which are illustrated by dashed line borders. For example, the diaper pant 100P may comprise a first color zone CZ1 comprising a first color, the first color zone being defined by a garment facing surface of the first waistband 200, and wherein the first color is defined by colorants in the waistband substrate 212. The diaper pant 100P may also comprise a second color zone CZ2 comprising a second color, the second color zone being defined by the garment facing surface of the first belt 106 in the chassis overlap region 852, and wherein the second color is defined by colorants in the first nonwoven substrate 162, the second nonwoven substrate 164, and the backsheet 136. In some configurations, the first nonwoven substrate 162 may comprise a first plurality of fibers having a first fiber color; and the second nonwoven substrate 164 may comprise a second plurality of fibers having a second fiber color, wherein the first fiber color and the second fiber color are different. The diaper pant 100P may also comprise a third color zone CZ3 defined by the garment facing surface of the first belt 106 in the first extensible region 853a and/or the second extensible region 853b. In some configurations, the third color zone CZ3 may also be defined by the garment facing surface of the first belt 106 extending laterally between the first side seam 178 and the second side seam 180 and longitudinally between the chassis overlap region 852 and the first waistband 200. In some configurations, the third color zone CZ3 may comprise a third relaxed color defined by colorants in the first nonwoven substrate 162 and the second nonwoven substrate 164 when the first belt 106 is in a fully relaxed state in the first extensible region 853a and/or the second extensible region 853b. The diaper pant 100P may also comprise a fourth color zone CZ4 comprising a fourth color, the fourth color zone defined by a garment facing surface 134 of the backsheet 136 extending longitudinally between the first belt 106 and the second belt 108. The fourth color zone CZ4 may comprise a fourth color defined by colorants in the backsheet nonwoven substrate 136 and the backsheet film substrate 136b.

As previously mentioned, diaper pants 100P may comprise solid colored nonwoven substrates and/or film substrates combined so as to provide relatively stark color contrasts between waistbands 200, 202 and belts 106, 108 and relatively subtle color contrasts between absorbent chassis 102 and belts 106, 108. Color differences between colors of color zones on diaper pant 100P components such as shown in FIG. 6 may be measured with the Color Measurement Method provided herein. For example, with reference to the description above and to FIG. 6, the first color and the second color may be different and may comprise a delta E of at least 10, measured according to the Color Measurement Method. In some configurations, the fourth color and the third relaxed color may comprise a delta E of less than 2.7, measured according to the Color Measurement Method.

### Examples

The following data and examples are provided to help illustrate the color variances within diaper pants as described herein. The exemplified structures are given solely for the purpose of illustration and are not to be construed as limitations of the present disclosure, as many variations thereof are possible without departing from the spirit and scope of the invention.

Table 1 below provides a summary of various delta E measurements made on example diaper pants according to the Color Measurement Method.

TABLE 1

Delta E Values

| | First Color | Third Relaxed Color |
|---|---|---|
| | versus | versus |
| | Second Color | Fourth Color |
| Example 1 | 56.6 | 5.7 |
| Example 2 | 19.4 | 5.7 |
| Example 3 | 13.7 | 2.7 |

### Example 1

Example 1 is an absorbent article with elastic laminate belt, waistband and chassis overlap region.

The ELASTIC LAMINATE BELT comprises the following materials and construction.

First nonwoven substrate: The first nonwoven substrate is a spunbond nonwoven with total basis weight of 17 gsm. The nonwoven is produced on a production line having 2 spin beams, where the fibers of the first and second beams comprise polypropylene, and a navy blue-colored masterbatch at a loading level up to 5 percent by weight and the pantone used for color is 2187U. The fibers have an average diameter ranging from 10 to 19 microns. The fibers are calender bonded using a bond pattern having S-shaped bonds and approximately 12-14% bond area.

Second nonwoven substrate: The second nonwoven substrate is a 15 gsm navy blue polypropylene nonwoven comprising produced at on a production line. The fibers have an average diameter ranging from 16 to 19 microns. The nonwoven is produced on a production line and comprise polypropylene, and a navy blue-colored masterbatch at a loading level up to 5 percent by weight and the pantone used for the color is 2187U.

The elastic laminate is produced using the above described first nonwoven substrate on the garment-facing side and the second nonwoven substrate on the body facing side. White elastic strands (Hyosung TNC Corporation 78 DTex, 300 N) are spaced at 1.6 mm and extended to 150% strain and are coated with adhesive (Bostik 4376, Bostik, Wauwatosa, WI) at a weight of 6 gram per square meter and adhered between the first and second nonwoven substrates.

### Waistband:

The waistband comprises a third nonwoven substrate which is a spunbond nonwoven produced at PFNonwovens, LLC (Hazleton, PA). The total basis weight of the nonwoven substrate is 25 gsm. The nonwoven is produced on a production line and a green colored masterbatch at a loading level up to 5 percent by weight and the pantone used for the color is 2426U. The fibers are calender bonded using a bond pattern having S-shaped bonds and approximately 14% bond area. The third nonwoven substrate was folded twice and 10 elastics (INVISTA Lycra® T837 540DTex elastic strands) are positioned between the first portion and the second portion of the third nonwoven substrate which are spaced at 3 mm and extended to 225% strain and are coated with adhesive (Bostik H2401 NF EA Omnimelt, Bostik, Wauwatosa, WI) at a weight of 18 milligrams per meter.

BACKSHEET REGION: The Example 1 absorbent article backsheet comprises a backsheet nonwoven substrate bonded with a backsheet film substrate. Backsheet nonwoven comprises the first nonwoven substrate from laminate belt assembly where the first nonwoven substrate is a spunbond nonwoven with total basis weight of 17 gsm. The nonwoven is produced on a production line having 2 spin beams, where the fibers of the first and second beams comprise polypropylene, and a navy blue-colored masterbatch at a loading level up to 5 percent by weight and the pantone used for the color is 2187U. The fibers have an average diameter ranging from 10 to 19 microns. The fibers are calendar bonded using a bond pattern having S-shaped bonds and approximately 12-14% bond area.

The backsheet film substrate is a thin breathable thermoplastic polymeric film having a thickness of about 0.012 mm to about 0.051 mm. This polymeric film is a polypropylene based low breathable film with basis weight of 15 gram per square meter and pantone used for the navy blue color is 7692U. This backsheet film substrate is bonded with backsheet nonwoven substrate with adhesive (Bostik H2401 NF EA Omnimelt, Bostik, Wauwatosa, WI) at a weight of 6 milligrams per meter.

### Example 2

Example 2 is an absorbent article with elastic laminate belt, waistband and chassis overlap region.

The ELASTIC LAMINATE BELT comprises the following materials and construction.

First nonwoven substrate: The first nonwoven substrate is a spunbond nonwoven with total basis weight of 17 gsm. The nonwoven is produced on a production line having 2 spin beams, where the fibers of the first and second beams comprise polypropylene, and a classic blue-colored masterbatch at a loading level up to 5 percent by weight and the pantone used for color is 2935U. The fibers have an average diameter ranging from 10 to 19 microns. The fibers are calender bonded using a bond pattern having S-shaped bonds and approximately 12-14% bond area.

Second nonwoven substrate: The second nonwoven substrate is a 15 gsm classic blue polypropylene nonwoven comprising produced at on a production line. The fibers have an average diameter ranging from 16 to 19 microns. The nonwoven is produced on a production line and comprise polypropylene, and a classis blue-colored masterbatch at a loading level up to 5 percent by weight and the pantone used for the color is 2935 U.

The elastic laminate is produced using the above described first nonwoven substrate on the garment-facing side and the second nonwoven substrate on the body facing side. White elastic strands (Hyosung TNC Corporation 78 DTex, 300 N) are spaced at 1.6 mm and extended to 150% strain and are coated with adhesive (Bostik 4376, Bostik, Wauwatosa, WI) at a weight of 6 gram per square meter and adhered between the first and second nonwoven substrates.

### Waistband:

The waistband comprises a third nonwoven substrate, which is a spunbond nonwoven produced at PFNonwovens, LLC (Hazleton, PA). The total basis weight of the nonwoven substrate is 25 gsm. The nonwoven is produced on a production line and a green colored masterbatch at a loading level up to 5 percent by weight and the pantone used for the color is 2426U. The fibers are calendar bonded using a bond pattern having S-shaped bonds and approximately 14% bond area. The third nonwoven substrate was folded twice and 10 elastics (INVISTA Lycra® T837 540DTex elastic strands) are positioned between the first portion and the second portion of the third nonwoven substrate which are spaced at 3 mm and extended to 225% strain and are coated with adhesive (Bostik H2401 NF EA Omnimelt, Bostik, Wauwatosa, WI) at a weight of 18 milligrams per meter.

BACKSHEET REGION: The Example 2 absorbent article backsheet comprises a backsheet nonwoven substrate bonded with a backsheet film substrate. The backsheet nonwoven comprises the first nonwoven substrate from the laminate belt assembly where the first nonwoven substrate is a spunbond nonwoven with total basis weight of 17 gsm. The nonwoven is produced on a production line having 2 spin beams, where the fibers of the first and second beams comprise polypropylene, and a navy blue-colored masterbatch at a loading level up to 5 percent by weight and the pantone used for the color is 2935 U. The fibers have an average diameter ranging from 10 to 19 microns. The fibers are calendar bonded using a bond pattern having S-shaped bonds and approximately 12-14% bond area.

The backsheet film substrate is a thin breathable thermoplastic polymeric film having a thickness of about 0.012 mm to about 0.051 mm. This polymeric film is a polypropylene based low breathable film with basis weight of 15 gram per square meter and pantone used for the navy blue color is 2143C. This backsheet film substrate is bonded with backsheet nonwoven substrate with adhesive (Bostik H2401 NF EA Omnimelt, Bostik, Wauwatosa, WI) at a weight of 6 milligrams per meter.

### Example 3

Example 3 is an absorbent article with elastic laminate belt, waistband and chassis overlap region.

The ELASTIC LAMINATE BELT comprises the following materials and construction.

First nonwoven substrate: The first nonwoven substrate is a spunbond nonwoven with total basis weight of 15 gsm. The nonwoven is produced on a production line having 2 spin beams, where the fibers of the first and second beams comprise polypropylene, and a denim blue-colored masterbatch at a loading level up to 5 percent by weight and the pantone used for color is 2216U. The fibers have an average diameter ranging from 10 to 19 microns. The fibers are calender bonded using a bond pattern having S-shaped bonds and approximately 12-14% bond area.

Second nonwoven substrate: The second nonwoven substrate is a 15 gsm classic blue polypropylene nonwoven comprising produced at on a production line. The fibers have an average diameter ranging from 16 to 19 microns. The nonwoven is produced on a production line and comprise polypropylene, and a classis blue-colored masterbatch at a loading level up to 5 percent by weight and the pantone used for the color is 2216U.

The elastic laminate is produced using the above described first nonwoven substrate on the garment-facing side and the second nonwoven substrate on the body facing side. White elastic strands (Hyosung TNC Corporation 78 DTex, 300 N) are spaced at 1.6 mm and extended to 150% strain and are coated with adhesive (Bostik 4376, Bostik, Wauwatosa, WI) at a weight of 6 gram per square meter and adhered between the first and second nonwoven substrates.

### Waistband:

The waistband comprises a third nonwoven substrate, which is a spunbond nonwoven produced at PFNonwovens, LLC (Hazleton, PA). The total basis weight of the nonwoven substrate is 25 gsm. The nonwoven is produced on a production line and a black colored masterbatch at a loading level up to 5 percent by weight and the pantone used for the color is Black6U. The fibers are calendar bonded using a bond pattern having S-shaped bonds and approximately 14% bond area. The third nonwoven substrate was folded twice and 10 elastics (INVISTA Lycra® T837 540DTex elastic strands) are positioned between the first portion and the second portion of the third nonwoven substrate which are spaced at 3 mm and extended to 225% strain and are coated with adhesive (Bostik H2401 NF EA Omnimelt, Bostik, Wauwatosa, WI) at a weight of 18 milligrams per meter.

BACKSHEET REGION: The Example 3 absorbent article backsheet comprises a backsheet nonwoven substrate bonded with a backsheet film substrate. Backsheet nonwoven is using the first nonwoven substrate from laminate belt assembly where the first nonwoven substrate is a spunbond nonwoven with total basis weight of 15 gsm. The nonwoven is produced on a production line having 2 spin beams, where the fibers of the first and second beams comprise polypropylene, and a denim blue-colored masterbatch at a loading level up to 5 percent by weight and the pantone used for the color is 2216U. The fibers have an average diameter ranging from 10 to 19 microns. The fibers are calendar bonded using a bond pattern having S-shaped bonds and approximately 12-14% bond area.

Backsheet film substrate is a thin breathable thermoplastic polymeric film having a thickness of about 0.012 mm to about 0.051 mm. This polymeric film is a polypropylene based low breathable film with basis weight of 15 gram per square meter and pantone used for the navy blue color is 7545C. This backsheet film substrate is bonded with backsheet nonwoven substrate with adhesive (Bostik H2401 NF EA Omnimelt, Bostik, Wauwatosa, WI) at a weight of 6 milligrams per meter.

### Color Measurement Method

Color within a zone on an absorbent article sample is measured using a handheld spectrophotometer, such as the X-rite exact Spectrophotometer (available from X-Rite, Grand Rapids MI), or an equivalent instrument, with 45°/0° geometry and a 4.0 mm optical aperture. Samples are conditioned at about 23° C. ±2 C.° and 50%±2% relative humidity for 2 hours before testing and all measurements are made under the same conditions.

Calibrate the instrument per the manufacturer's instructions with the following settings: CIE L*a*b* units, 2° Observer, C Illumination, Abs White Base, no Physical Filter, and the Density Standard of ANSI T.

The testing zone on the sample is identified (see for example the color zones described above with reference to FIG. 6) and backed with a standard white tile. The spectrophotometer is then placed on a site within the testing zone, such that the instrument's aperture is located entirely within the testing zone. A color reading is made according to the instrument manufacturer's instructions, and the CIE L*a*b* color values are recorded to the nearest 0.01 units. The measurement is repeated at a total of three (3) replicate sites within the testing zone. The average of the three L*, a*, and b* values within the zone is calculated and each is recorded to the nearest 0.01 units.

The Delta E (ΔE) color difference between zones is calculated by identifying the average

L*, a*, and b* values from a first zone as L*1, a*1, and b*1, and identifying the average L*, a*, and b* values from a second zone as L*2, a*2, and b*2. The Delta E value is then calculated according to the following equation:

$$Delta E = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}}$$

The Delta E value is then reported to the nearest 0.1 units.

### Combinations

A1. An absorbent article comprising: a first belt comprising a belt proximal edge and a belt distal edge longitudinally outboard of the proximal edge, and a first plurality of elastic strands positioned between and connected with a first nonwoven substrate and a second nonwoven substrate; a second belt, wherein laterally opposing end portions of the second belt are connected with laterally opposing end portions of the first belt at a first side seam and a second side seam; a chassis comprising a topsheet, a backsheet, and an absorbent core positioned between the topsheet and the backsheet, the chassis comprising a longitudinally extending first side edge and a longitudinally extending second side edge laterally separated from the first side edge by a first end edge and a second end edge longitudinally separate from the first end edge, and wherein longitudinally opposing end regions of the chassis are connected with the first belt and the second belt; wherein a portion of the backsheet overlaps an inner wearer facing surface of the first belt to define a chassis overlap region, and wherein the first belt comprises a first extensible region extending laterally between the chassis overlap region and the first side seam and a second extensible region extending laterally between the chassis overlap region and the second side seam; a waistband comprising a waistband distal edge and a waistband proximal edge, and a second plurality of elastic strands connected with a third nonwoven substrate, wherein a wearer facing surface of the waistband is connected with a garment facing surface of the first belt, wherein the waistband distal edge is positioned longitudinally outboard of the belt distal edge, and wherein the waistband proximal edge is positioned longitudinally inboard of the belt distal edge; a first color zone comprising a first color, the first color zone being defined by a garment facing surface of the waistband, and wherein the first color is defined by colorants in the third nonwoven substrate; and a second color zone comprising a second color, the second color zone being defined by the garment facing surface of the first belt in the chassis overlap region, and wherein the second color is defined by colorants in the first nonwoven substrate, the second nonwoven substrate, and the backsheet; and wherein the first color and the second color are different and comprise a delta E of at least 10, measured according to a Color Measurement Method.

A2. The absorbent article of paragraph A1, further comprising: a third color zone defined by the garment facing surface of the first belt in the first extensible region, wherein the third color zone comprises a third relaxed color when the first belt is in a fully relaxed state in the first extensible region.

A3. The absorbent article of paragraph A2, further comprising: a fourth color zone comprising a fourth color, the fourth color zone defined by a garment facing surface of the backsheet extending longitudinally between the first belt and the second belt, and wherein the fourth color and the third relaxed color comprise a delta E of less than 2, measured according to the Color Measurement Method.

A4. The absorbent article of any of paragraphs A1 to A3, wherein the first nonwoven substrate comprises a first plurality of fibers having a first fiber color; and wherein the second nonwoven substrate comprises a second plurality of fibers having a second fiber color, wherein the first fiber color and the second fiber color are different.

A5. The absorbent article of any of paragraphs A1 to A4, wherein the backsheet comprises a backsheet nonwoven substrate bonded with a backsheet film substrate.

A6. The absorbent article of paragraph A5, wherein the chassis overlap region comprises a first area wherein the backsheet nonwoven substrate is bonded with the inner wearer facing surface of the first belt and a second area wherein the backsheet film substrate is bonded with the inner wearer facing surface of the first belt.

A7. The absorbent article of paragraph A6, wherein some of the first plurality of elastic strands extend across the second area.

A8. The absorbent article of paragraph A7, wherein the first plurality of elastic strands do not extend across the first area.

A9. The absorbent article of paragraph A8, wherein the second area is positioned longitudinally between the first end edge of the chassis and the first area.

A10. The absorbent article of any of paragraphs A1 to A9, wherein the first plurality of elastic strands are spaced from each other by about 0.25 mm to about 4 mm and comprise a Dtex of from about 10 to about 500.

A11. The absorbent article of any of paragraphs A1 to A10, wherein the second plurality of elastic strands are spaced from each other by about 0.25 mm to about 4 mm and comprise a Dtex of from about 10 to about 500.

A12. The absorbent article of any of paragraphs A1 to A11, wherein the waistband is a laminate comprising: a first fold line in the third nonwoven substrate; a first portion of the third nonwoven substrate extending from the first fold line; a second portion of the third nonwoven substrate extending from the first fold line, the second portion in a facing relationship with the first portion; wherein the second plurality of elastic strands are positioned between the first portion and the second portion of the third nonwoven substrate.

A13. The absorbent article of paragraph A12, wherein the laminate further comprises: a second fold line in the third nonwoven substrate; a third portion of the third nonwoven extending from the second fold line and in a facing relationship with the first portion; and wherein the first portion extends between the first fold line and the second fold line.

A14. The absorbent article of paragraph A13, wherein the second fold line defines the waistband distal edge.

A15. The absorbent article of paragraph A13, wherein the third portion is connected with the garment facing surface of the first belt.

A16. The absorbent article of any of paragraphs A1 to A15, wherein the waistband and the first belt have matching textures.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a first belt comprising a belt proximal edge and a belt distal edge longitudinally outboard of the proximal edge, and a first plurality of elastic strands positioned between and connected with a first nonwoven substrate and a second nonwoven substrate;
a second belt, wherein laterally opposing end portions of the second belt are connected with laterally opposing end portions of the first belt at a first side seam and a second side seam;
a chassis comprising a topsheet, a backsheet, and an absorbent core positioned between the topsheet and the backsheet, the chassis comprising a longitudinally extending first side edge and a longitudinally extending second side edge laterally separated from the first side edge by a first end edge and a second end edge longitudinally separate from the first end edge, and wherein longitudinally opposing end regions of the chassis are connected with the first belt and the second belt;
wherein a portion of the backsheet overlaps an inner wearer facing surface of the first belt to define a chassis overlap region, and wherein the first belt comprises a first extensible region extending laterally between the chassis overlap region and the first side seam and a second extensible region extending laterally between the chassis overlap region and the second side seam;
a waistband comprising a waistband distal edge and a waistband proximal edge, and a second plurality of elastic strands connected with a third nonwoven substrate, wherein a wearer facing surface of the waistband is connected with a garment facing surface of the first belt, wherein the waistband distal edge is positioned longitudinally outboard of the belt distal edge, and wherein the waistband proximal edge is positioned longitudinally inboard of the belt distal edge;
a first color zone comprising a first color, the first color zone being defined by a garment facing surface of the waistband, and wherein the first color is defined by colorants in the third nonwoven substrate; and
a second color zone comprising a second color, the second color zone being defined by the garment facing surface of the first belt in the chassis overlap region, and wherein the second color is defined by colorants in the first nonwoven substrate, the second nonwoven substrate, and the backsheet; and
wherein the first color and the second color are different and comprise a delta E of at least 10, measured according to a Color Measurement Method.

2. The absorbent article of claim 1, further comprising:
a third color zone defined by the garment facing surface of the first belt in the first extensible region, wherein the third color zone comprises a third relaxed color when the first belt is in a fully relaxed state in the first extensible region.

3. The absorbent article of claim 2, further comprising:
a fourth color zone comprising a fourth color, the fourth color zone defined by a garment facing surface of the backsheet extending longitudinally between the first belt and the second belt, and
wherein the fourth color and the third relaxed color comprise a delta E of less than 2, measured according to the Color Measurement Method.

4. The absorbent article of claim 1, wherein the first nonwoven substrate comprises a first plurality of fibers having a first fiber color; and wherein the second nonwoven substrate comprises a second plurality of fibers having a second fiber color, wherein the first fiber color and the second fiber color are different.

5. The absorbent article of claim 1, wherein the backsheet comprises a backsheet nonwoven substrate bonded with a backsheet film substrate.

6. The absorbent article of claim 5, wherein the chassis overlap region comprises a first area wherein the backsheet nonwoven substrate is bonded with the inner wearer facing surface of the first belt and a second area wherein the backsheet film substrate is bonded with the inner wearer facing surface of the first belt.

7. The absorbent article of claim 6, wherein some of the first plurality of elastic strands extend across the second area.

8. The absorbent article of claim 7, wherein the first plurality of elastic strands do not extend across the first area.

9. The absorbent article of claim 8, wherein the second area is positioned longitudinally between the first end edge of the chassis and the first area.

10. The absorbent article of claim 1, wherein the first plurality of elastic strands are spaced from each other by about 0.25 mm to about 4 mm and comprise a Dtex of from about 10 to about 500.

11. The absorbent article of claim 1, wherein the second plurality of elastic strands are spaced from each other by about 0.25 mm to about 4 mm and comprise a Dtex of from about 10 to about 500.

12. The absorbent article of claim 1, wherein the waistband is a laminate comprising:
a first fold line in the third nonwoven substrate;
a first portion of the third nonwoven substrate extending from the first fold line;
a second portion of the third nonwoven substrate extending from the first fold line, the second portion in a facing relationship with the first portion;
wherein the second plurality of elastic strands are positioned between the first portion and the second portion of the third nonwoven substrate.

13. The absorbent article of claim 12, wherein the laminate further comprises:
a second fold line in the third nonwoven substrate;
a third portion of the third nonwoven extending from the second fold line and in a facing relationship with the first portion; and
wherein the first portion extends between the first fold line and the second fold line.

14. The absorbent article of claim 13, wherein the second fold line defines the waistband distal edge.

15. The absorbent article of claim 13, wherein the third portion is connected with the garment facing surface of the first belt.

16. The absorbent article of claim 1, wherein the waistband and the first belt have matching textures.
